(19) United States
(12) Patent Application Publication
(10) Pub. No.: US 2014/0207005 A1
(43) Pub. Date: Jul. 24, 2014

(54) SYSTEM AND METHOD FOR REAL-TIME THREE DIMENSIONAL MODELING OF CARDIOVASCULAR DYNAMICS AND THE HEART USING ELECTROCARDIOGRAM SIGNALS

(71) Applicant: The Board of Regents for Oklahoma State University, Stillwater, OK (US)

(72) Inventors: Satish Bukkapatnam, Stillwater, OK (US); Trung Le, Stillwater, OK (US); Ranga Komanduri, Stillwater, OK (US)

(21) Appl. No.: 14/163,894

(22) Filed: Jan. 24, 2014

Related U.S. Application Data
(60) Provisional application No. 61/756,243, filed on Jan. 24, 2013.

(51) Int. Cl. A61B 5/0245; A61B 5/02; A61B 5/00; A61B 5/021

(52) U.S. Cl. CPC A61B 5/0245; A61B 5/021; A61B 5/02007; A61B 5/742; A61B 5/7235
USPC 600/485; 600/509; 600/513

(57) ABSTRACT

There is taught herein a real-time, lumped parameter cardiovascular dynamics model that uses features extracted from online electrocardiogram (ECG) signal recordings to generate certain surrogate hemodynamic signals. The derived signals can be used to real time animate a 3-D heart model. The model represents the coupled dynamics of the heart chambers, valves, and pulmonary and systemic blood circulation loops in the form of nonlinear differential equations. The features extracted from ECG signals can be used to estimate the timings and amplitudes of the atrioventricular activation input functions as well as other model parameters that capture the effect of cardiac morphological and physiological characteristics. The results indicate the potential of a virtual instrument that uses the model-derived signals for clinical diagnosis in lieu of expensive instrumentation.

### CROSS REFERENCE TO RELATED APPLICATIONS

This application claims the benefit of U.S. Provisional Patent Application Ser. No. 61/756,243, filed on Jan. 24, 2013 and incorporates said provisional patent application by reference into this document as if fully set out at this point.

### STATEMENT REGARDING FEDERAL SPONSORED RESEARCH OR DEVELOPMENT

This invention was made with U.S. Government support under NSF Grant No. CMMI-0729552 and NSF Grant No. CMMI-0830023 awarded by the National Science Foundation. The Government has certain rights in this invention.

### FIELD OF THE INVENTION

This disclosure relates to medical diagnoses in general and, more particularly, the real-time three dimensional cardiovascular modeling.

### BACKGROUND

Cardiovascular diseases are a leading cause of mortality in the U.S. Delivery of cardiovascular healthcare, especially to rural and isolated communities, remains a major challenge despite billions spent annually on medical devices for cardiovascular diagnostics and treatment. The development of affordable and accessible medical instrumentation is essential for promoting early diagnosis, thereby reducing cardiovascular disease treatment costs.

Digital computers have made it possible to quantitatively model the complex mechanisms and interactions in the cardiovascular system. For example, the impedance characteristics of arterial segments have been employed in a lumped parameter fluid dynamics model of the circulation system. A difference-differential equation circulation system model based on segmenting the arterial flow along the axial and radial directions of a cylindrical coordinate system has also been utilized.

A seminal model by Guyton et al. (A. C. Guyton, et al., “Circulation: Overall regulation,” Annual Review of Physiology, vol. 34, pp. 13-44, 1972) used more than 350 compartments to analyze the underlying interactions and predict the circulation states of the cardiovascular system, the disclosure of the reference as it relates to heart models and/or circulation being incorporated herein by reference in full as if set out at this point. Subsequently, research efforts to simulate cardiovascular pathologies based on Guyton's model have been reported.

In many of these lumped parameter models, atrioventricular activation functions can be characterized effectively in terms of a time-varying elastance e(t) that describes the average instantaneous variation of pressure (P) for a unit change in the volume (V) of an atrium (a) or ventricle (v), i.e.,

$${e}_{a , v} ( t ) = {〈 \frac{\partial P}{\partial V} 〉}_{a , v} .$$

Normalized e(t) of the left ventricle has been shown to be fairly independent of loading conditions, contractile state, and the heart rate.

Subsequently, it has been shown that P-V characteristics are nonlinear during the ejection phase of a cardiac cycle, when intraventricular pressures exceed the aortal and pulmonary arterial pressures to propel blood out of the ventricles, as well as in the isovolumic relaxation phase, when the intraventricular pressures decrease rapidly to cause the aortic and pulmonary valves to abruptly close. A method has also been presented that estimates the end-systolic elastance of the left ventricle from a single cardiac cycle. Doppler-echocardiography and radio-nuclide ventriculography has been used to capture P-V characteristics at every heart-beat. Earlier approaches to estimating e(t) used data collected from expensive instrumentation under well-defined conditions from ex vivo or in vivo experiments on an animal (e.g., dog or rat) heart, or a specified group of patients.

Analytical lumped parameter and/or computational (e.g., finite element) modeling of the complex interactions among electrical, mechanical, and chemical processes that underpin cardiovascular dynamics is important to the development of a virtual cardiovascular instrument (“VCP”). Lumped parameter models offer lower computational cost and a straightforward physical interpretation of the dynamic interactions among the elements of a cardiovascular system.

Mathematical model-based platforms such as LiDCOplus™ and Picco2™ are used in critical care settings to provide estimates of blood pressure, stroke volume variations, and cardiac output. However, these platforms rely on invasive arterial pulse pressure measurements to estimate hemodynamic signals. They are, therefore, limited in their applications for easy bedside monitoring across a range of medical settings.

Therefore, the derived elastance curves tend to have limitations for capturing the real-time beat-to-beat and inter-subject variations in e(t) characteristics.

Additionally, it is well known that current methods of 3D real-time heart modeling and display are not entirely satisfactory. Three-dimensional heart modeling is a relatively new focus in the field of computer imaging and cardiology with the majority of literature dating back only for the last 20 years. The field of cardiology focuses on non-invasive diagnosis and treatment and, as a last resort, surgery. Improving computer technologies and processors have enabled advanced visualization and processing in many fields. Motivations for developing a 3D heart model fall into three general categories: finite element mesh (FEM) models, computer animation, and biomechanics.

One approach creates adaptive and quality 3D meshes from volumetric image data such as Computed Tomography (CT), Magnetic Resonance Imaging (MRI) and Signed Distanced Function (SDF). Another approach utilizes an FEM model that is developed from echocardiography, i.e., in one instance a FEM model was generated in real time from transophogeal echocardiography. Methods of smoothing mechanical contractions and interactions between the four chambers have been achieved successfully utilizing implicit surfaces. However, heretofore animation of the mechanical movement of the heart has been limited to static replications of a beating heart. Previous work does not incorporate live signals to drive the animation of the heart either in real-time or processed and exported in an animated movie format. Different aspects of animating cardiac activity have been achieved through surface ECG mapping and VCG display. One approach has been to use a beat dynamic electrical heart. One such model was able to detect mechanical movement from cardiac electrical activity. However, the purpose of this model was to improve existing ECG readings rather than drive a mechanical animation of the heart

As is well known in the medical diagnosis arts, there has been a very real need for a system and method that would address and solve the above-described problems.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY

There is taught herein a real-time, lumped parameter cardiovascular dynamics model that uses features extracted from online electrocardiogram (ECG) signal recordings to generate certain surrogate hemodynamic signals which can be used, in turn, to produce model-derived signals for clinical diagnosis in lieu of expensive instrumentation.

According to an embodiment, a lumped parameter model to relate the states of a cardiovascular system to ECG signal features has been developed. Analogous to the virtual instruments used in engineering measurement practice, in one embodiment the model utilizes a single channel of the ECG suite of leads to generate multiple virtual hemodynamic signals including pressure, volume, respiratory impedance, and blood flow rate. Lead number two (II) has been found suitable for use in some embodiments.

In one embodiment, the model represents the coupled dynamics of the heart chambers, valves, and pulmonary and systemic blood circulation loops in the form of nonlinear differential equations. The features extracted from ECG signals can be used to estimate the timings and amplitudes of the atrioventricular activation input functions as well as other model parameters that capture the effect of cardiac morphological and physiological characteristics. Test results suggest that the model can capture the salient time and frequency patterns of the measured central venous pressure, pulmonary arterial pressure, and respiratory impedance signals.

One embodiment utilizes a method based on the Anderson-Darling statistic and Kullback-Leibler divergence to compare the clinical measures (i.e., systolic and diastolic pressures) estimated from model waveform-extrema with those from actual measurements. The test statistics of the model waveform-extrema were statistically indistinguishable from the measured values with beat-to-beat rejection rates of 10%. The results indicate the potential of a virtual instrument that uses the model-derived signals for clinical diagnosis in lieu of expensive instrumentation.

In some embodiments the derived signals are used to real time animate a 3-D heart model. The model represents the coupled dynamics of the heart chambers, valves, and pulmonary and systemic blood circulation loops in the form of nonlinear differential equations. The features extracted from ECG signals can be used to estimate the timings and amplitudes of the atrioventricular activation input functions as well as other model parameters that capture the effect of cardiac morphological and physiological characteristics. According to an embodiment, a finite element numerical scheme might be used to construct a model that represents the heart. That being said, this is just one of many possible numerical schemes (e.g., finite difference, etc.) that could be used.

According to still another embodiment there is provided a real time display of a model of the heart that is particularized to a particular person. More particularly, there is provided herein an embodiment that uses a single lead of an ECG to drive a model of that patient's heart which includes such parameters

The foregoing has outlined in broad terms the more important features of the invention disclosed herein so that the detailed description that follows may be more clearly understood, and so that the contribution of the instant inventors to the art may be better appreciated. The instant invention is not limited in its application to the details of the construction and to the arrangements of the components set forth in the following description or illustrated in the drawings. Rather the invention is capable of other embodiments and of being practiced and carried out in various other ways not specifically enumerated herein. Additionally, the disclosure that follows is intended to apply to all alternatives, modifications and equivalents as may be included within the spirit and the scope of the invention as defined by the appended claims. Further, it should be understood that the phraseology and terminology employed herein are for the purpose of description and should not be regarded as limiting, unless the specification specifically so limits the invention.

### BRIEF DESCRIPTION OF THE DRAWINGS

Other objects and advantages of the invention will become apparent upon reading the following detailed description and upon reference to the drawings in which:

FIG. 1 contains one approach to generating certain surrogate hemodynamic waveforms from measured ECG features.

FIG. 2 contains an exemplary extraction of P, T loop and QRS complex from VCG.

FIG. 3 illustrates atrioventricular activation functions synthesized from ECG features.

FIG. 4 shows a summary of an ECG-based parameter estimation method for a virtual cardiovascular model.

FIG. 5 illustrates an overall implementation diagram and zoomed out left atrial block of a virtual cardiovascular model.

FIG. 6 contains waveforms that represent the pressure (P) of left atrium and left ventricle (left), right atrium and right ventricle (right).

FIG. 7 contains waveforms that represent the volume (V) of left atrium and left ventricle (left), right atrium and right ventricle (right).

FIG. 8 illustrates a comparison between (left) time and (right) frequency portraits of model derived (solid line) and measured (dashed line) pulmonary arterial pressures.

FIG. 9 contains a comparison of (left) time and (right) frequency portraits of model-derived right atrial pressure (Pᵣₐ, solid line) and the measured central venous pressure (CVP, dashed line).

FIG. 10 contains a comparison of the waveforms of the model-derived pulmonary venous pressure Pₚᵥ and the measured respiratory impedance, RI.

FIG. 11 contains KL convergence variations of systole and diastole pressures between model outputs and actual measurements.

FIG. 12 contains a block diagram of the left ventricle elastance for an embodiment.

FIG. 13 contains a block diagram of the pressure and volume in the left ventricle for an embodiment.

FIG. 14 illustrates an embodiment of a block diagram block of the aortic valve.

FIG. 15 contains a block diagram of the systemic aortic sinus segments.

FIG. 16 contains a block diagram of systemic artery segment.

FIG. 17 contains a block diagram of systemic vein segment.

FIG. 18 contains an illustration of an ECG curve and corresponding heart volumes and pressures calculated therefrom.

FIG. 19 contains a schematic illustration of Anterior MI rigid weighting regions of a heart model.

FIG. 20 contains a schematic operating logic suitable for use with an embodiment of the instant invention.

FIG. 21 is a schematic representation of a finite element mesh of a heart.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

In various embodiments of the present disclosure, a virtual cardiovascular instrument (VCI) has been developed in which the multiple data sets necessary for clinical diagnostics are generated through transformation of one measured signal. This approach can obviate the need for expensive clinical diagnostic instrument suites and, thus, has the potential to alleviate healthcare cost and access issues.

Some embodiments of the invention include an ability to:
(a) use a single ECG lead to derive atrioventricular activation functions based on relating the measured ECG signal events to the functions of various aspects of the cardiovascular processes;
(b) estimate the parameters of a cardiovascular dynamics model so that real-time rendering of the hemodynamic signals from the measured ECG is facilitated; and
(c) test the similarity of clinically relevant systolic and diastolic pressures extracted from the model to those from actual measurements.

Experimental investigations suggest that this sort of model can capture the salient time and frequency patterns of certain measured hemodynamic signals (e.g., central venous pressure, pulmonary arterial pressure, and respiratory impedance) and provide real-time estimates of systolic and diastolic pressures—the key indicators in clinical practice. In some embodiments, VCIs capable of generating certain hemodynamic signals relevant for diagnostics without the need for expensive instrumentation and/or invasive clinical procedures will be possible.

Turning now to a discussion of an embodiment, certain temporal intervals and amplitudes of the recorded ECG and ECG-derived respiration signals were used to estimate the parameters of e(t) in real time. Such ECG feature combinations are known to be unique to an individual's cardiovascular system. Therefore, the ECG-derived parameters of e(t) tend to capture the effects of physiological differences among individual cardiovascular systems.

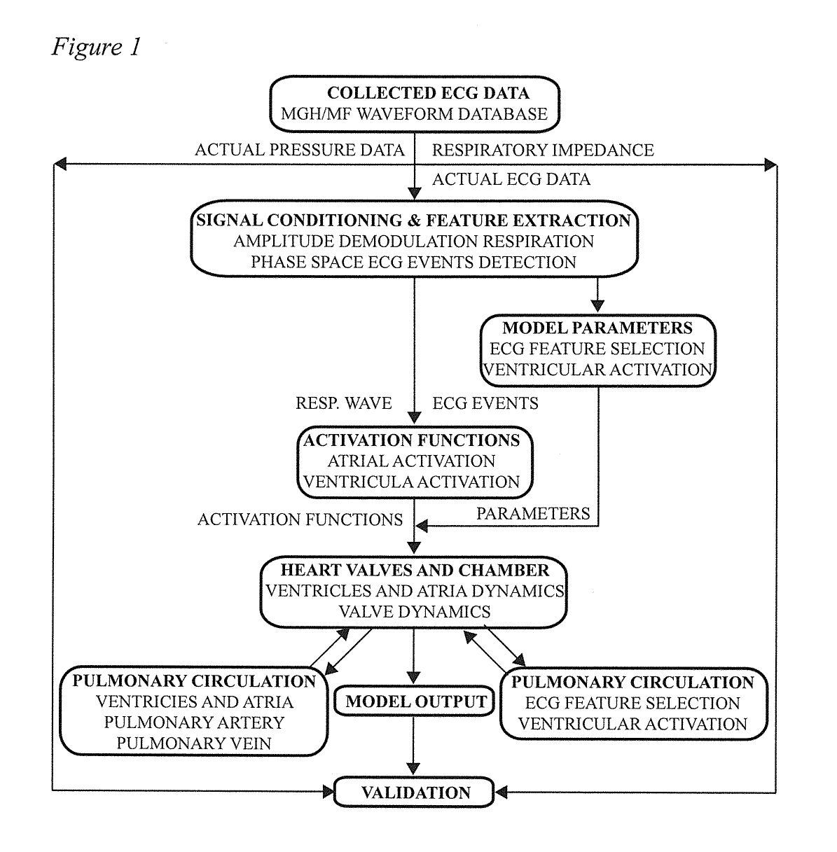

One inventive modeling approach to generating certain surrogate hemodynamic waveforms from measured ECG features is summarized in FIG. 1. The dataset from PhysioNet's MGH/MF waveform database was used. Twenty recordings were selected (subjects in the 42-84-year age range) in critical care settings with a variety of medical conditions. Each recoding consisted of synchronous measurements of ECG (lead-II), central venous pressure (CVP), pulmonary arterial pressure (PAP), and respiratory impedance (RI) gathered over 12-86 min duration at 360 Hz sampling rate. To reduce the simulation runtime and nonstationarity effects, signals gathered from each subject were partitioned into 60 second segments. According to one embodiment, the approach integrates the following four tasks:
(A) Signal conditioning and feature extraction: Respiratory components and ECG events (e.g., the peaks of P, R, and T waves and the offset of the T waves) were extracted from lead II of the ECG to formulate the activation functions. A phase space reconstruction method was utilized to extract critical events from the measured ECG signal.
(B) Cardiovascular model formulation: The model in this embodiment is based on extending previous cardiovascular dynamics models through the use of activation functions obtained from ECG features as excitation inputs.
(C) Parameter estimation: The salient model parameters according to this embodiment were estimated in real time using a multiple regression model with a compact set of ECG features selected as independent variables. The regression model coefficients were estimated offline in this particular case.
(D) Model validation: The model was validated by comparing the time and frequency patterns of the outputs with those of the recordings from the database.
(E) Real Time 3D Heart Model Animation. The chosen model was implemented in real time using the calculated elastance functions and an ECG signal.

A. Signal Conditioning and Feature Extraction

A1) De-Noising and Respiration Extraction:

Low frequency components (0-0.05 Hz) of raw ECG signals are mostly associated with wandering baseline anomalies, and high frequencies (>80 Hz) with the noise from the ambient environment, measurement devices, and other artifacts. In this example, a band-pass filter with a 0.06-40 Hz pass band was employed to remove these extraneous components and retain the ECG features vital for respiration and fiducial point extraction.

Further with respect to this example, an amplitude demodulation method was employed to extract the respiration signal Resp(t) from the ECG. Here, a pulse series was formed with the local average of RR interval values as the pulse intervals; the series was load-modulated by the corresponding R amplitudes. The respiration signal Resp(t) was extracted from linear interpolation of the resulting pulse series.

A2) ECG Event Detection:

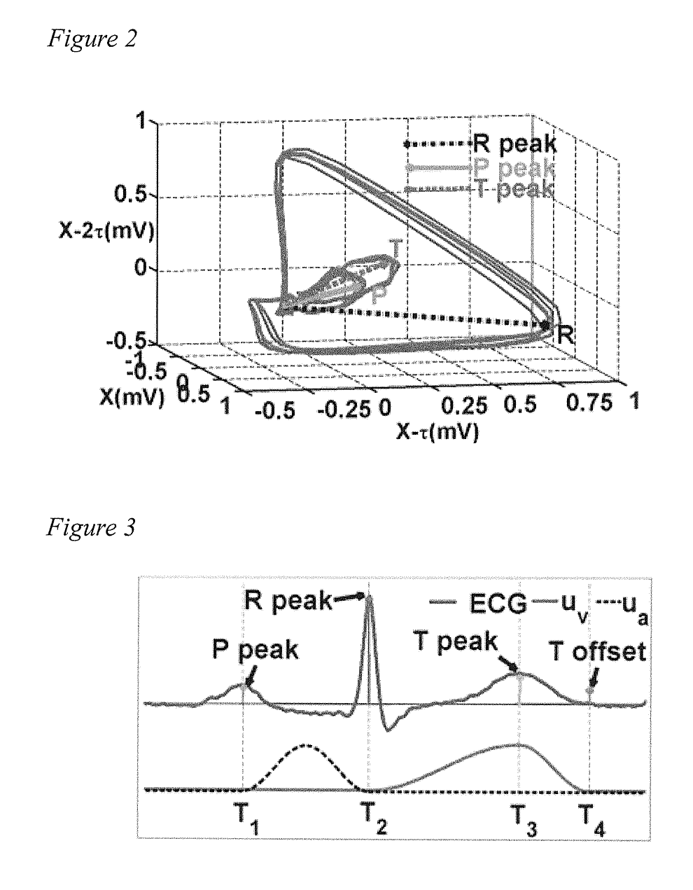

Continuing with the present example, first, a wavelet filter was used to detect R peaks in the de-noised ECG signal. Next, a vector time series was obtained by embedding the de-noised ECG signal in a three-dimensional state space with 12 ins time-delay. As shown in FIG. 2, the trajectories in the reconstructed state space portray three loops—the smallest loop (marked with the green solid line) captures predominantly the P-wave behavior; the largest (the black dotted line), the QRS complex; and the third (the red dashed line) captures the T wave characteristics. The maximum vector magnitudes in the P and T loops were taken to locate the peaks of the P and T waves. The peaks of the Q and S waves were identified at the minimum points in the time domain in PR interval and RT interval, respectively, in the de-noised ECG signal. The J point, defined as the junction of the QRS complex and the ST segment, was designated as the first inflection point (location where ECG waveform changes from concave to convex) after the S peak. The offset of the T wave was set at J+80 ms if the heart rate (HR) was less than 100 beat/min, J+72 if 100≦HR≦110, J+64 if 110≦HR≦120, and J+60 if HR>120. The extracted ECG events thus were used to derive the time profiles of the activation functions and the covariates for a regression model to estimate the model parameters.

B. Cardiovascular Model Formulation

B1) Activation Functions:

One cardiovascular model uses the elastance characteristics of the four heart chambers and the events extracted from the ECG signal to construct the activation (excitation) functions. Electromechanical delays are neglected because they tend to be a fraction of the signal sampling intervals as well as the time scales of the features (>0.1 sec) considered in this study. In an embodiment, the same activation function (uₐ) for both atria and an identical activation function (uᵥ) for both ventricles was used.

In an embodiment the activation function of the atria (see FIG. 3), whose support is assumed to be contemporaneous with the atrial systole period, takes the form of the following raised cosine function:

$${u}_{a} ( t ) = \{ \begin{matrix}0.5 \times ( 1 - cos ( \frac{2 π ( t - {T}_{1} )}{( {T}_{2} - {T}_{1} )} ) ) & {T}_{1} \leq t \leq {T}_{2} \\ 0 & t ∉ ( {T}_{1} , {T}_{2} )\end{matrix}$$

In this particular example the onset, T₁, and the offset, T₂, of the atrial systole are marked as the times of P peak and R peak events, respectively.

Two raised cosine functions were used to approximate the ventricle activation function during the isovolumic contraction, ejection, and relaxation processes (which take place between the end diastole T₂ and end systole T₄ of a cardiac cycle) as:

$${u}_{v} ( t ) = \{ \begin{matrix}0.5 \times ( 1 - cos ( \frac{π ( t - {T}_{2} )}{{T}_{3} - {T}_{2}} ) ) & {T}_{2} \leq t \leq {T}_{3} \\ 0.5 \times ( 1 + cos ( \frac{π ( t - {T}_{3} )}{{T}_{4} - {T}_{3}} ) ) & {T}_{3} < t \leq {T}_{4} \\ 0 & t ∉ ( {T}_{2} , {T}_{4} )\end{matrix}$$

where, in this example, T₂, T₃, and T₄ are assumed to be synchronous with R peak, T peak, and T offset event, respectively (see FIG. 3). Accordingly, in this embodiment the ventricular activation starts at the R peak, reaches the maximum amplitude at the T peak (the beginning of the ventricular ejection), and subsides at the offset of the T wave.

B2) Cardiovascular System:

One model was adapted to capture the dynamics of the four heart chambers, namely, right and left ventricles and atria and the two systemic and pulmonary circulations of the cardiovascular system. According to the current example all four sets of equations for the chambers assumed a similar form. For example, the dynamics of left ventricle volume (Vₗᵥ) and pressure (Pₗᵥ) are given by:

$$\frac{d {V}_{lv}}{d t} = {CQ}_{ao} {N}_{ao} \sqrt{\vert {P}_{lv} - {P}_{sas} \vert}$$

$${P}_{lv} = {P}_{lv , 0} + {e}_{lv} ( {V}_{lv} - {V}_{lv , 0} )$$

where Nₐₒ depends on the angular position of the aortic valve leaflets θₐₒ as:

Nₐₒ=(1−cos θₐₒ)²/(1−cos θ_{max,ao})²

The elastance functions for the ventricles and atria can be expressed in terms of the an embodiment of the inventive ECG-derived activation functions for the left ventricle and atrium as:

eₗₐ(t)=E_{lad}+(Eₗₐₛ−E_{lad})*(uₐ(t)+γResp(t))

eₗᵥ(t)=E_{lvd}+(Eₗᵥₛ−E_{lvd})*(uᵥ(t)+γResp(t)),

with corresponding equations for the right ventricle and atrium given by:

eᵣₐ(t)=E_{rad}+(Eᵣₐₛ−E_{rad})*(uₐ(t)+γResp(t))

eᵣᵥ(t)=E_{rvd}+(Eᵣᵥₛ−E_{rvd})*(uᵥ(t)+γResp(t)).

In the foregoing, Eₗᵥₛ, E_{lvd}, Eₗₐₛ, E_{lad} capture the amplitude (gain) of the elastance waveforms, and γ quantifies the coupling strength of the respiratory effect on the cardiovascular system activation beyond constricting and dilating the activation functions in the previous equations based on changes in heart rate. The definition and calculation of the parameters Eₗᵥₛ, E_{lvd}, Eₗₐₛ, E_{lad} and γ are well known to those of ordinary skill in the art and need not be presented herein.

In one embodiment ventricular elastance depends on the free wall, pericardium, and thoracic chamber pressures. Assuming the environmental pressure to be invariant and the body movement to be negligible, the intrathoracic chamber pressure increases during expiration, concomitant with the chest/rib cage contraction, and decreases during inspiration, concomitant with the chest expansion. The instantaneous elastance is thus influenced by respiration. Since the respiration signal Resp(t) aggregates the aforementioned expansions and contractions, the coupling parameter γ was used to quantify the (first order) sensitivity of the elastance functions to respiration, and hence the thoracic chamber pressure.

Aortic valve dynamics can be expressed as:

$$\frac{{d}^{2} {θ}_{ao}}{d {t}^{2}} = ( {P}_{lv} - {P}_{sas} ) {K}_{p , ao} cos {θ}_{ao} - {K}_{f , ao} \frac{d {θ}_{ao}}{d t} + {K}_{b , ao} {Q}_{ao} cos {θ}_{ao} - {K}_{v , ao} {Q}_{ao} ( μ ( {Q}_{ao} ) sin {θ}_{ao} + μ ( - {Q}_{ao} ) cos {θ}_{ao} )$$

where μ(•) is a unit step function and the remainder of the symbols are listed in the Appendix.

The systemic circulation loop in some variations consists of the aortic sinus, artery, arteriole, capillary, and vein segments, and each individual component is modeled by considering the local blood flow resistance, the elasticity of blood vessels, and the inertia of blood. The systemic pressures and flow rates can be given by:

$$\{ \begin{matrix}\frac{d {P}_{sas}}{d t} = \frac{{Q}_{ao} - {Q}_{sas}}{{C}_{sas}} ; \\ \frac{d {Q}_{sas}}{d t} = \frac{{P}_{sas} - {P}_{sat} - {R}_{sas} {Q}_{sas}}{{L}_{sas}} ; \\ \frac{d {P}_{sat}}{d t} = \frac{{Q}_{sas} - {Q}_{sat}}{{C}_{sat}} \\ \frac{d {Q}_{sat}}{d t} = \frac{{P}_{sat} - {P}_{svn} - ( {R}_{sat} + {R}_{sar} + {R}_{scp} ) {Q}_{sas}}{{L}_{sat}} ; \\ \frac{d {P}_{svn}}{d t} = \frac{{Q}_{sat} - {Q}_{svn}}{{C}_{svn}} ; \\ {Q}_{svn} = \frac{{P}_{svn} - {P}_{ra}}{{R}_{svn}}\end{matrix}$$

The variation of pressures and flow rates across the pulmonary loop can be quantified using similar function forms. Altogether, the cardiovascular process dynamics are expressed in terms of deterministic nonlinear differential equations with the parameters and activation functions estimated from the measured ECG.

C. ECG-Based Parameter Estimation

In one embodiment the model parameters include the elastance characteristics of the left and right ventricles (i.e., Eₗᵥₛ, Eᵣᵥₛ, E_{lvd}, and E_{rvd}), the respiratory coupling γ, and six other parameters, namely, Gₚₐ, Oₚₐ, Gᵣₐ, Oᵣₐ, Gₚᵥ, and Oₚᵥ, that quantify the gain and offset of the pulmonary arterial pressure (Pₚₐ), right atrial pressure (Pᵣₐ), and pulmonary venous pressure (Pₚᵥ) signals. Quantification of these parameters requires complex, invasive measurements taken over several conditions. Note that, for purposes of the instant disclosure, the foregoing parameters will be collectively referred to as cardiovascular hemodynamic parameters.

Although the foregoing cardiovascular hemodynamic parameters are not typically used for patient evaluation/diagnosis and embodiments of the instant invention are primarily intended to use same for purposes of real time heart model animation, it should be noted that it would be possible to use these parameter values (which will tend to be constant for an individual) in a screening or diagnostic sense. As an example of how they might be used, consider the starting values for these parameter values listed in the Appendix to this disclosure, the contents of which are fully incorporated by reference as if set out at this point. These starting values were chosen to be equal to established norms for the associated parameter. Since the calculated parameters relate directly to heart properties, a diagnosing physician could utilize the calculated values for an individual by comparing them with an average population value or norm. Obviously, if one or more parameters deviate from the norm such deviation would at least raise a concern that more thorough testing might be called for. Of some importance is that these parameters are easily obtainable according to an embodiment without invasive tests or expensive imaging machines.

In this embodiment an offline statistical model was developed that maps these model parameters to appropriate ECG features. Embodiments of the present method are able to adaptively estimate the model parameters and is more responsive to anatomical and physiological characteristics of individual subjects as well as real-time physiological changes as captured in ECG patterns compared to conventional parameter selection methods.

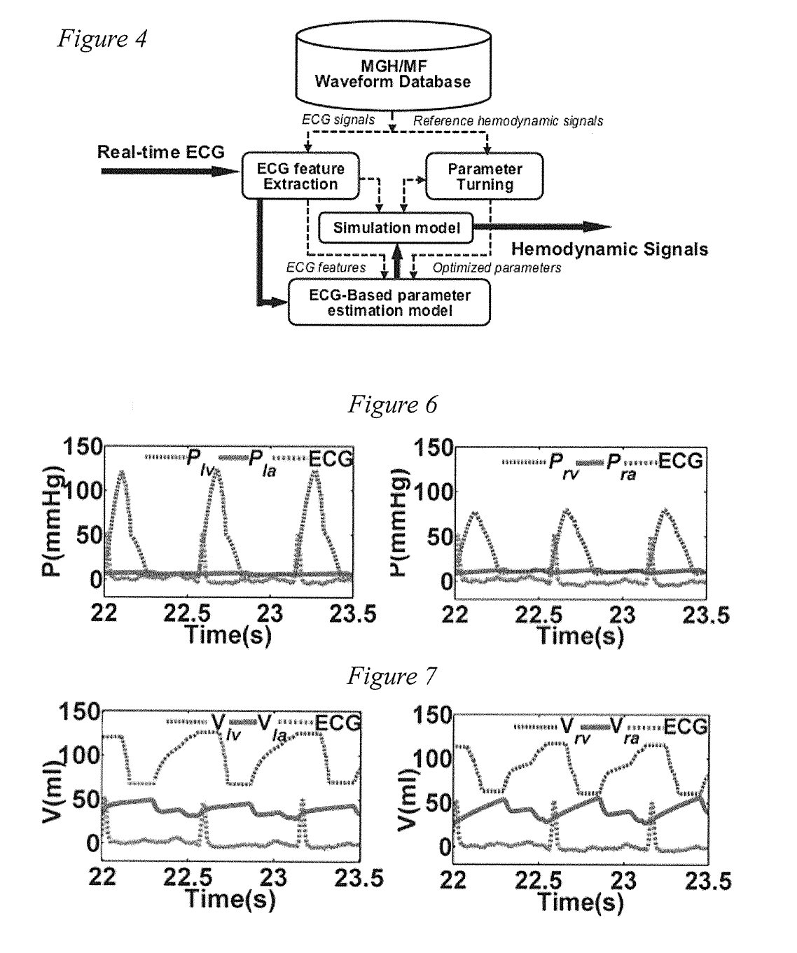

FIG. 4 describes the training (dashed line) and simulation (solid line) phases of the virtual cardiovascular model of one embodiment. According to this example the training phase consists of building an empirical multiple regression model to estimate a set of model parameters from the selected ECG features. The simulation phase consists of generating certain hemodynamic signals from the cardiovascular model whose parameters are estimated from online ECG features using the trained regression model.

C1) Parameter Tuning:

One purpose of parameter tuning is to determine an optimized model parameter set that minimizes the mean square error between the simulation model outputs and the measurements from recording signals. In this variation, the model-estimated pressure signals Pᵣₐ and Pₚₐ are compared with measured central venous pressure (CVP) and pulmonary arterial pressure (PAP) signals, respectively. The vector of tuned parameters of this variation consists of Gₚₐ, Oₚₐ, Gᵣₐ, Oᵣₐ, Gₚᵥ, Oₚᵥ, γ, Eₗᵥₛ, Eᵣᵥₛ, E_{lvd}, and E_{rvd}. A global optimization-based pattern search algorithm was used to find the optimized parameters. The optimization problem can be defined as follows:

min f(x)_{xεΩ}s.t l≦Ax≦u

where f: Rⁿ→R is the objective function, xεRⁿ, l, uεR^{m} and AεQ^{m×n}.

The pattern search algorithm utilized in one embodiment consists of two phases: the search step and the poll step. Initially, xₖ is assigned as the starting point and Δₖ is the coarseness of the grid Mₖ defined over Ω. In the search step, the objective function is evaluated at a finite number of points on the mesh Mₖ. If f(xₖ₊₁)<f(xₖ), the search step is considered successful; then the mesh is coarsened to Δₖ₊₁≧Δₖ, and the search step is restarted from the improved point xₖ₊₁. If an improved point is not found on the mesh, the poll step is invoked by considering the initial points that are the neighbors of xₖ on the mesh. Objective functions at the neighboring mesh points are evaluated to see if a lower function value can be found. If an improved mesh point is found (i.e., f(xₖ₊₁₎<f(xₖ)), Δₖ₊₁>Δₖ is set, and the search is restarted from the improved point. Otherwise, the search is started with mesh refinement Δₖ₊₁<Δₖ. Multiple (>20) starting points have been used to minimize the probability that the algorithm will converge to a local minimum.

C2) ECG-Based Parameter Estimation Model:

According to an embodiment, the following four groups were identified with a total of 15 ECG features that can collectively track much of the subject-to-subject variations in anatomical and physiological characteristics to estimate the salient model parameters: (i) sample averages of P, QRS, T peak amplitudes, (ii) sample averages of RR, PR, ST, QT intervals, (iii) standard deviations and differences between intervals, and (iv) the area swept by the ST segment. These features were selected based studies that have shown that an ECG trace expresses cardiac features unique to an individual. From a physiological standpoint, changes in R and T amplitudes in frontal and precordial leads are associated with instantaneous changes in the volume and output of the left ventricle. Specifically, the R amplitude is sensitive to the radial movement of the heart (dilation) in relation to the chest wall and the T wave amplitude to the variations in the ratio of the endocardial to the epicardial surface area of the left ventricle.

The heart rate (estimated from RR interval) is determined in this embodiment by the vagal-sympathetic mechanisms, and the systolic period (estimated from intra-beat intervals of ECG) is affected by the sympathetic efferent discharge frequency (F_{con}), which in turn influences the peak elastance]. A rise in F_{con} increases the maximum elastance and shortens the ventricular systole. Also, it is known that short term fluctuations of heart rates are affected by the autonomic control levels at pacemaker sites. It has been shown that low frequency heart rate fluctuations provide an index of sympathetic efferent activity, which are associated with the peak ventricular elastance. The standard deviations of the ECG intervals (RR, ST, PR, QT intervals) can therefore be used to capture the effects of vagal and sympathetic neural activities, and hence contribute to the estimation of the peak ventricular elastance.

In this embodiment the selected features, extracted via phase space analysis described below, were used as predictors for a regression model to derive model parameters. It may be noted that many ECG features contain redundant information, and each model parameter tends to be sensitive to a different combination of features. Principal component analysis (PCA) was used to address these redundant and diverse relationships. The subsets of ECG features whose contributions to the principal components are the largest were selected for parameter estimation.

To predict the parameters of the cardiovascular model according to this embodiment, a multiple linear regression model φ=Xβ+ε was considered, where φ is an n×1 vector of model parameters, X is an n×p full-column rank matrix of predictors (i.e., ECG features), β is a p×1 vector of unknown regression model coefficients, and ε is an independent Gaussian random variable. A backward stepwise procedure was used to select the most predictive feature combinations. The procedure begins with considering all variables in the model and sequentially deleting one variable at a time. A partial F-test for each variable in the presence of the others was conducted with the test statistic

$$F = \frac{( {β }^{⋒} {X}^{T} φ - n {\overline{φ}}^{2} ) / p}{{φ}^{T} φ - {β}^{⋒} {X}^{T} φ / ( n - p - 1 )} \sim {F}_{p , n - p - 1} .$$

The stepwise procedure continues until the smallest F has p-value >0.05. A k-fold cross validation was used to assess for the generalizability of the regression model. A random 90% of the dataset was selected for fitting the multiple regression model and the remaining 10% for validation. This process was repeated 20 times to assess the consistency of the regression coefficients.

D. Model Validation

While the model-generated pressure waveforms can be compared with those from the actual recordings, only certain extreme values of the waveforms are considered clinically important. For example, many of the clinical assessment procedures for a subsequent coronary heart disorder use the extrema (systolic and diastolic) blood pressure waveforms. Therefore, one needs a test procedure that weights the waveform conformance according to the clinical importance of the specified portion or pattern of the waveform. According to this embodiment, the Anderson-Darling goodness of fit test was used to measure the similarity of the systolic and diastolic pressures between the model outputs and the actual measurements. A two-sample Anderson-Darling statistic is defined as

$${A}_{nm}^{2} = \frac{nm}{N} {\int }_{- ∞}^{∞} \frac{{[ {F}_{n} ( x ) - {G}_{m} ( x ) ]}^{2}}{{H}_{N} ( x ) [ 1 - {H}_{N} ( x ) ]} d {H}_{N} ( x )$$

$${H}_{N} ( x ) = [ {nF}_{n} ( x ) + {mG}_{m} ( x ) ] / N$$

where Fₙ (x), Gₘ (x) are the empirical distribution functions of the systolic and diastolic pressures of actual measurements and the model derived signals, and H_{N} (x) is the empirical distribution function of the combined sample size (N=n+m). The distinct values in the combined data set, ordered from smallest to largest, are denoted as z₁, z₂, . . . z_{N}. According to this aspect of invention, the systolic and diastolic blood pressure values extracted from the model waveform were compared to those from the measurements. As stated in the foregoing, the Anderson-Darling statistic is believed to be more appropriate for use in the present context than the other two-sample statistics (e.g., K-S test, Cramer-von Mises) because it places more weight on observations in the tails of the distribution through the use of the weight function [H(x)(1−H(x))]⁻¹. The test statistic can be estimated from a sample (zⱼ) as:

$${A}^{2} = \frac{n + m - 1}{{( n + m )}^{2}} \times [ \frac{1}{n} {\sum }_{j = 1}^{N} \frac{{{h}_{j} ( ( n + m ) {F}_{j} - {nH}_{j} )}^{2}}{{H}_{j} ( n + m - {H}_{j} ) - \frac{( n + m ) {h}_{j}}{4}} += \frac{1}{m} {\sum }_{j = 1}^{N} \frac{{{h}_{j} ( ( n + m ) {G}_{j} - {mH}_{j} )}^{2}}{{H}_{j} ( n + m - {H}_{j} ) - \frac{( n + m ) {h}_{j}}{4}} ]$$

where hⱼ is the number of observations of the combined sample equal to zⱼ, Hⱼ is the number of values in the combined samples with values less than zⱼ plus one half the number of values in the combined samples equal to zⱼ, and Fⱼ and Gⱼ are the sample sizes of the measured and model-derived signals, respectively, that are less than zⱼ plus one half the number of values in that group that are equal to zⱼ.

Under the null hypothesis that F(x) and G(x) are drawn from the same distribution, the variance of the test statistics A² is given by:

$${σ}^{2} = var ( {A}^{2} ) = \frac{{aN}^{3} + {bN}^{2} + cN + d}{( N - 1 ) ( N - 2 ) ( N - 3 )}$$

where a, b, c, d are derived from F. W. Scholz and M. A. Stephens, “K-sample Anderson-Darling tests,” Journal of the American Statistical Association, vol. 82, 1987. If the test statistic A² is above a critical value given by ADC=1+1.961σ, then one can reject (at significance level α=0.05) the hypothesis that the measurements and the model-derived signals were drawn from the same distribution.

In addition, Kullback-Leibler (KL) divergence [55] was used to assess the closeness of the systolic and diastolic pressure distributions of the model outputs to the actual measurements. KL divergence is given by:

D_{KL}(g;f)=Σᵢ₌₁^{k}g(xᵢ)log{g(xᵢ)/f(xᵢ)}

where f, g are the density functions of the measured and the model-derived signal, respectively. As KL divergence D_{KL}(g; f) is an asymmetric measurement, we used M_{KL}=[D_{KL}(g; f)+D_{KL}(f; g)]/2 as a symmetrized metric to compare the distribution similarity. The larger the KL divergence value, the farther apart are the two signals. The KL divergence value equals zero when the two distributions are identical.

E. Real Time 3D Heart Model Animation

According to another embodiment, there is provided a method of providing a real-time three-dimensional heart model with animation capabilities. Inputs into the model include ECG signals (either 3-lead VCG, 12-lead ECG or simply a single lead that will be transformed using an affine transformation matrix). In some embodiment, the inputs are processed by the integrated back-end, the activation and lump heart dynamic model. The model of this embodiment uses the Simulink toolbox to process ECG and generate individual heart chamber (left ventricle, right ventricle, left atrium, and right atrium) volumes, pressures, valve dynamics, and heroic flow rates. These variables serve as inputs to the 3D heart model.

E1) Base Heart Model Development

It is import to use an anatomically and physiologically accurate heart model in this embodiment. A finite element method (FEM) mesh model (e.g., FIG. 21) developed by Zhang (i.e., Zhang, Y., C. Bajaj, and B. Sohn, 3D finite element meshing from imaging data, Computer Methods □in Applied Mechanics and Engineering, 2005) was used in one embodiment, the disclosure of this document being incorporated in its entirety herein for all purposes as if set out fully at this point. This model was selected for the following characteristics: physiologically accurate structure, individual chambers, inner and outer surfaces, and high-resolution valves. Clearly, this is just one sort of model that might be utilized and those of ordinary skill in the art will readily be able to select other suitable model types.

According to this embodiment, the mesh connectivity for the model is comprised of triangular nodes. The Matlab® trisurf function plots X, Y, and Z coordinates according to a connectivity matrix having dimensions of n by 3. This function also allows alpha transparency specifications. This option makes it possible to view allows the inner surface to be seen which may be useful in some embodiments.

E2) Transparent Heart Model Integration with VCG

VCG offers additional data and perspective in comparison to 12-lead ECG. By taking the magnitude of leads X, Y, and Z, the VCG loop can be plotted. VCG demonstrates spatial information and conductive pathways of cardiac electrical activity.

E3) Heart Animation—Separating Chambers

According to this embodiment, the model supplied has indices for location of each individual point (valves, inner chambers, etc.) but the outer surface is represented as one structure. It was necessary to separate points into left atrium, right atrium, left ventricle, and right ventricle.

Piecewise dividing lines were utilized to separate the heart into quadrants with each chamber occupying only one quadrant. This was done by sorting points individually into four individual matrices (LA, LV, RA, RV) as well a matrix for the aorta. Points that did not correspond to that matrix had (0,0,0) inputted in its place. Otherwise, the location of the point was appended to the matrix. This method was chosen to quickly be able to adjust each chamber independently while maintaining the ability to add all chambers together for each point in time during the animation.

All=LA+LV+RA+RV+aorta

When using the existing connectivity mesh, all five parts can be meshed together with minimal processing.

E4) Heart Animation—Animation Mechanism

The animation in this embodiment relies on manipulating the left aorta, left ventricle, right aorta, right ventricle and aorta individually. The models then are scaled based on the backend simulation from the instantaneous volumes generated. The relaxed or uncontracted form of heart is based on the maximum volume of a beat. For real-time applications, this can be done by utilizing an initialization beat before displaying real-time information.

Continuing with the present example, the scalar for contraction can be found by the following equation:

L=Max−V−Min
L=Volume Scalar
Max=Maximum chamber volume
Min=Minimum chamber volume
V=Instantaneous chamber volume
   Point contraction can be accomplished through the use of polar coordinates.

This in turn is used in the actual contraction of points.

$$R = {R}_{0} - {R}_{0} * ( \frac{L}{abs ( sin ( 2 * PHI ) )} )$$

where,
R=New point radius
R₀=Original point radius
PHI=Point altitude angle.
   This particular arrangement allows the actual volumes to drive the reduction in volume size. However, the polar coordinate contraction method produces various boundary issues. This is resolved through several mechanisms discussed in Model Smoothing below.

E5) Heart Animation—Resampling the Mesh

Depending on the computational resources available, resampling the mesh may be necessary to perform calculations and post processing on the model. One embodiment of the original model contained 50,000 nodes and the associated computations required computational power that slows down even modern computers. The model was processed using a nearest neighbor resampling to reduce the mesh by a factor of 25. For the simulation, a model with 2000 nodes was utilized with no loss in structure or anatomical accuracy.

E6) Heart Animation—Model Smoothing

One approach to smoothing the model would be to utilize a simple average. In this particular approach, the previous frame, current frame, and next frame were averaged to get an interpolated plot. This proved to show some success. However, this embodiment of the model requires a more robust model to adapt to various conditions such as myocardial infarction.

F. Weight Matrix

Myocardial infarction (“MI”) is one of the most severe and prevalent forms of heart disease. Occurrence of an MI causes dead tissue from a blockage and lack of blood supply to certain regions of the heart. When cardiac tissue dies, it can no longer contract and that region essentially becomes rigid. In this embodiment, assigning a rigidity coefficient to each point on the heart is used to account for “stiffness” in that region.

According to an embodiment, a method of assigning a rigidity constraint to each point on the heart is presented that successfully solves boundary condition issues and refines the model. Nodes that are more rigid and do not move as much can be assigned a lower coefficient value. On that same token, nodes on the ventricles can be assigned a higher value. In the case of myocardial infarction where tissue has died and does not move to the same degree as healthy material, a rigidity coefficient for each point would allow the model to demonstrate the effects of MI or other conditions.

F1) Myocardial Infarction Weights

For purposes of illustration only, Anterior Myocardial Infarction will be used as an example. Turning to FIG. 19, the region shown within oval 1910 has been assigned a high rigid value. Three additional surrounding regions (shown approximately in FIG. 19 as ovals 1920, 1930, and 1940) have different rigid coefficient values to allow for smoothing between regions and to accurately model a beating heart with anterior MI. This configuration enables the model heart to better demonstrate the effects of MI on the mechanical motion. The center of the infarction is selected as the center of the region 1910. In the present example, each region 1910-1940 has decreasing rigidness relative to the region inside of it.

F2) Smooth Gradient

To improve on existing polar contraction animation mechanisms, in an embodiment applying weights along the chambers may provide a smoother mechanism. In this particular case, weights will be chosen to either increase or decrease the contraction amount of each point as the point is farther from the center of the heart. This overcomes many of the artifacts associated with uniform contraction in a non-uniform volume.

Using weighting, a model's boundary issues can be handled by applying a weighting gradient over the each region. This will help to overcome “cliffs” or uneven drop-offs between ventricles and atria.

F3) MI Weighting of Infracted Region

Integrating all of the weighting factors, this model can visually demonstrate MI. To further demonstrate the MI model's differences the affected MI region can be colored black, for example, to represent necrotic tissue. This method enhances visualization of MI in addition to the mechanical adjustments.

G. Cardiovascular Physiological Calculations

Cardiologists and other physicians use a number of calculated figures to diagnose cardiovascular conditions. As an example, physicians often use volumetric data from the heart to calculate stroke volume, ejection fraction and cardiac output.

In order to calculate these three fractions, end-diastolic volume (EDV) and end-systolic volume (ESV) are useful. These can be derived from echocardiogram data, for example. EDV of the left ventricle is the maximum volume of the chamber each beat, and as its name suggests, is the volume at the end of diastole. ESV is the end of systole volume for the left ventricle. The stroke volume is the volume pumped (displacement) from one chamber each beat. In this embodiment, the stroke volume can be calculated simply as:

SV=EDV−ESV

The ejection fraction is the EDV that is ejected each beat and can be calculated as:

$$\begin{matrix}{E}_{f} = \frac{SV}{EDV} \\ = \frac{EDV - ESV}{EDV}\end{matrix}$$

The last ratio above can be defined as the Cardiac Output (CO). The cardiac output is the volumetric flow rate of pumped blood by the heart. It is calculated by:

CO=HR×SV,

where HR is heart rate and SV is stroke volume.

All three of the previous quantities can be useful in gauging the health of the heart and how efficiently it is pumping. Review of these quantities provides useful information to supplement the physician's understanding of a patient's cardiovascular system.

Utilizing the above methods it is possible to animate a dynamic beating heart that adjusts in real time to the ECG signal that is fed into it.

The implementation of the model and all derivatives of it are to utilize the beating mechanism in a real-time environment. Ideally a device with VCG will be utilized but even standard 12-lead ECG machines with digital signals can be used by transforming the signal in real-time from 12-lead to VCG using a linear transformation matrix.

H. Implementation Details and Results

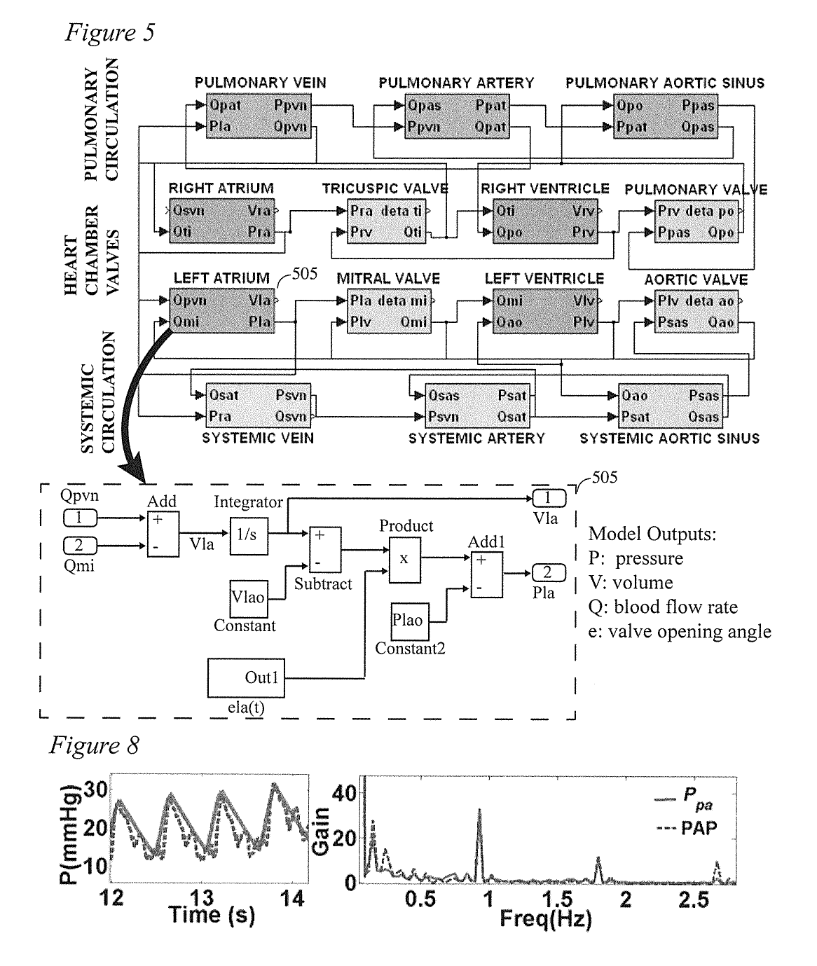

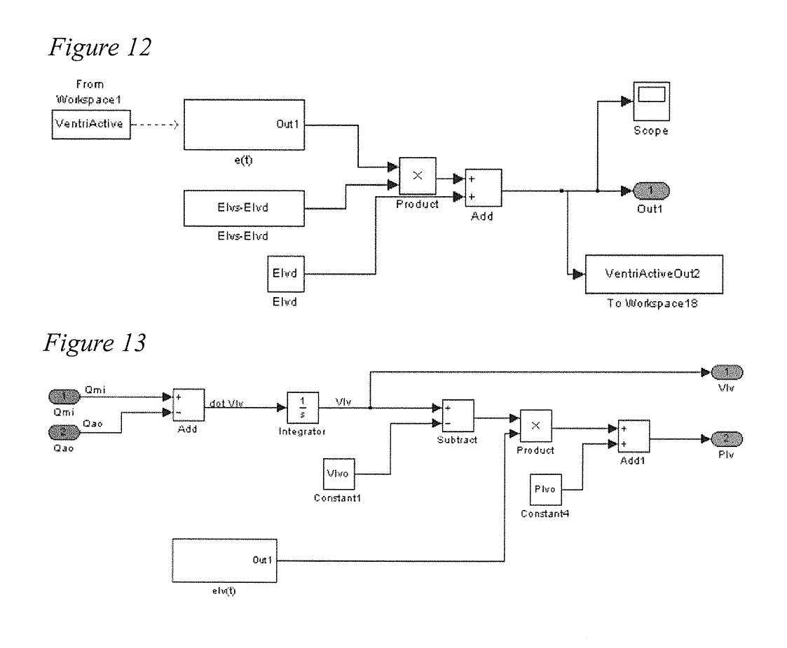

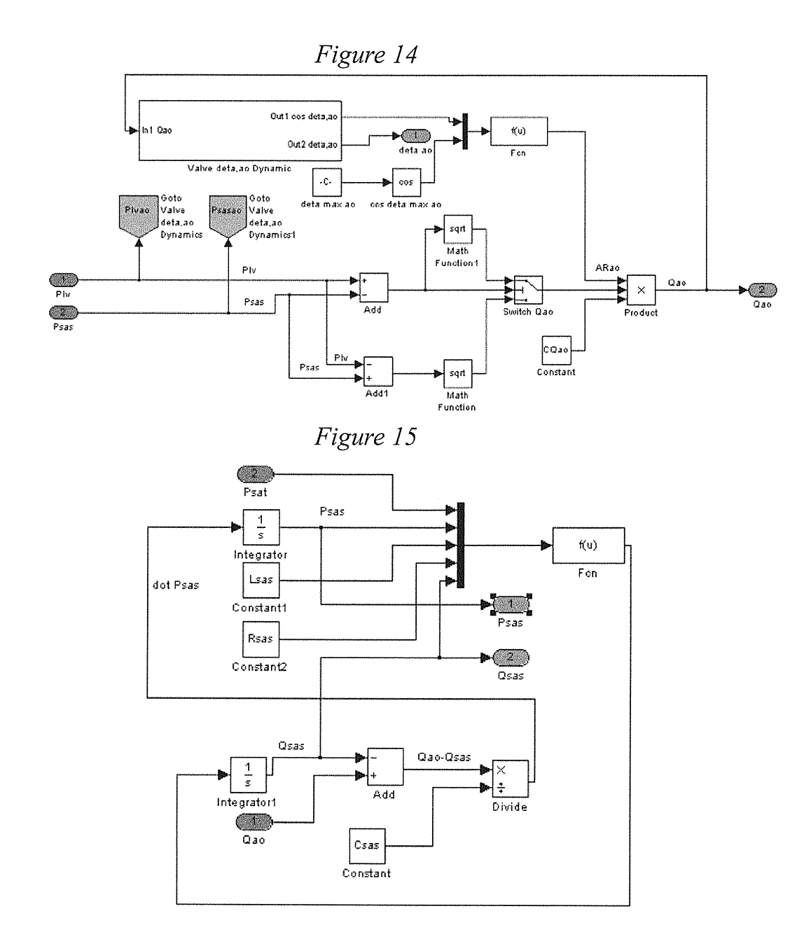

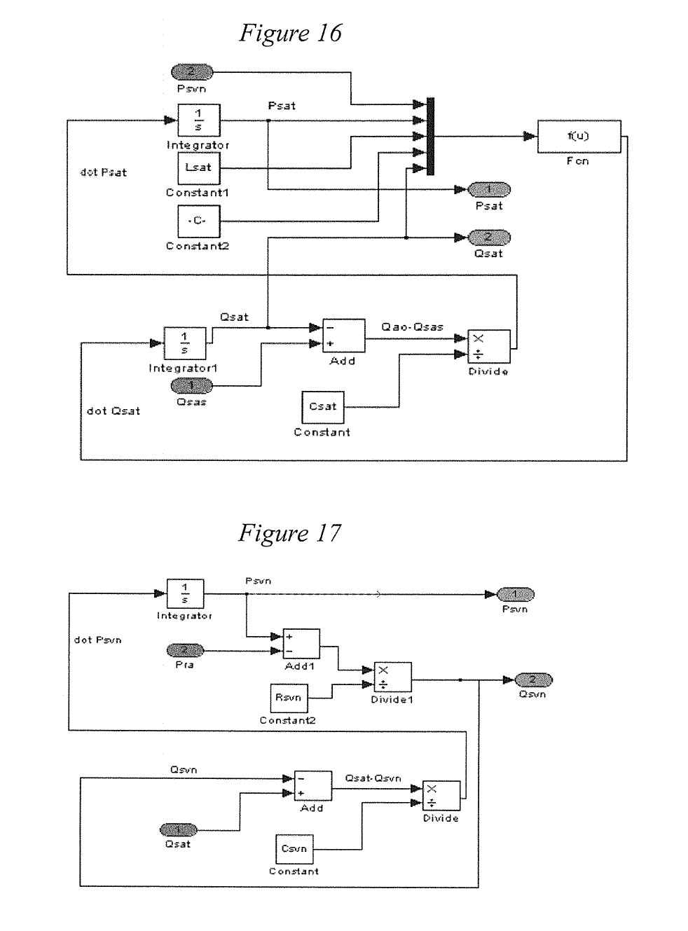

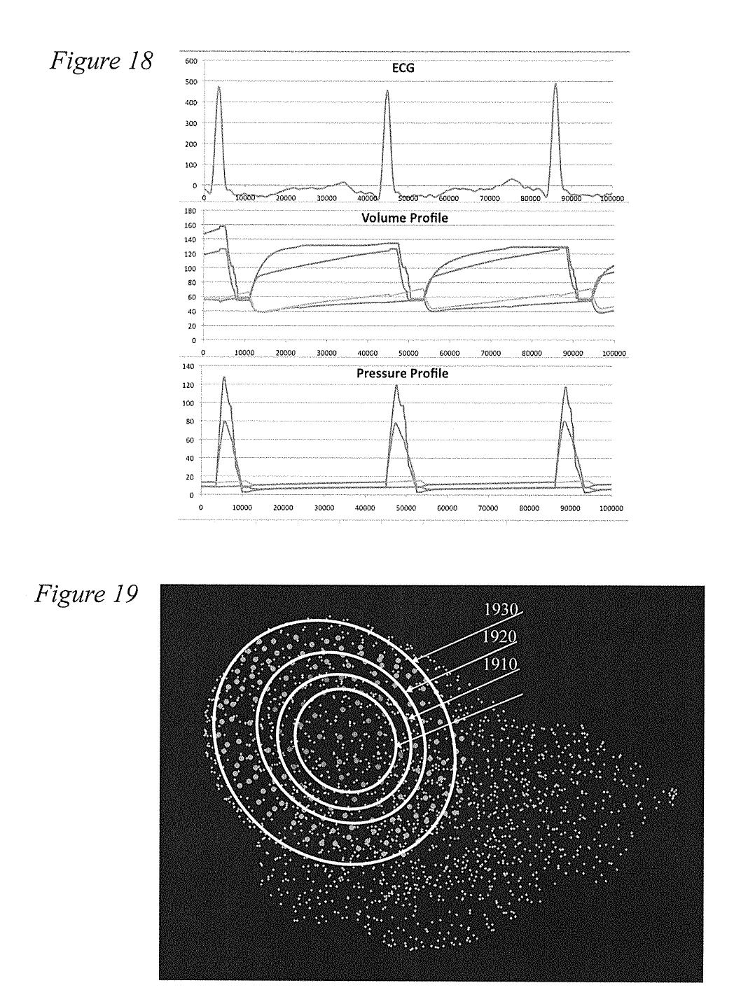

According to this embodiment the model was implemented in the Matlab/Simulink environment (see FIG. 5). Additional details of the embodiment of FIG. 5 can be found in the expanded view of block 505 within FIG. 5, in FIG. 12 (which contains an expanded view of the subsystem of the 2 ventricles where the elastance functions are introduced to the model), within FIG. 13 (which contains an expanded view of the left ventricle block with inputs Qmi, and Qao, and outputs are Vlv and Plv, wherein in each block the input quantifiers are annotated with the black arrows, the other quantifiers are the outputs), FIG. 14 (which contains an expanded view of the aortic block with inputs and outputs as indicated), FIG. 15 (which contains an expanded view of the systemic aortic sinus block with inputs and outputs as indicated), FIG. 16 (which contains an expanded view of the systematic artery block with inputs and outputs as indicated) and FIG. 17 (which contains an expanded view of the systemic vein block with inputs and outputs as indicated).

The sample times of the signal sources and the model were matched so that the outputs of the model were synchronized with the input ECG signal. Every simulation was run for 60 seconds. The transient effects were noted to subside by the end of 10 seconds. Therefore, data collected after 10 seconds of the simulation were used for further analysis. The model outputs consist of waveforms of the pressure, volume, flow rate from the four heart chambers, and the pulmonary and systemic circulation modules.

TABLE 1
CONTRIBUTION OF ECG FEATURES TO
THE FIRST FOUR PRINCIPAL COMPONENTS

| Feature | Description (Unit) | Comp 1 | Comp 2 | Comp 3 | Comp 4 |
|---|---|---|---|---|---|
| R AmpAvg | Average of R peak amplitudes | −0.03 | 0.36 | −0.10 | 0.27 |
| | (mV) | | | | |
| P AmpAvg | Average of P peak amplitudes | −0.26 | 0.26 | 0.00 | −0.41 |
| | (mV) | | | | |
| T AmpAvg | Average of T peak amplitudes | −0.10 | 0.35 | −0.08 | 0.37 |
| | (mV | | | | |
| RR Avg | Average of RR intervals (s) | 0.31 | 0.15 | −0.25 | −0.01 |
| PR Avg | Average of PR intervals (s) | −0.10 | 0.12 | −0.04 | −0.45 |
| ST Avg | Average of ST intervals (s) | 0.42 | 0.05 | 0.07 | −0.21 |
| QT Avg | Average of QR intervals (s) | 0.42 | 0.03 | 0.11 | −0.24 |
| RR Std | Standard deviation of RR | −0.03 | 0.16 | 0.09 | −0.27 |
| | intervals (s) | | | | |
| PR Std | Standard deviation of PR | 0.28 | 0.17 | −0.12 | 0.40 |
| | intervals (s) | | | | |
| ST Std | Standard deviation of ST | 0.05 | −0.11 | −0.65 | −0.11 |
| | intervals (s) | | | | |
| QT Std | Standard deviation of QT | 0.05 | −0.10 | −0.65 | −0.11 |
| | intervals (s) | | | | |
| ST-PR | ST, PR interval differences (s) | 0.23 | −0.02 | 0.08 | 0.05 |
| QT-PR | QT, PR interval differences (s) | 0.44 | −0.02 | 0.12 | −0.01 |
| ST Area | Average of ST segment area | −0.01 | 0.54 | 0.05 | −0.02 |
| | (mV s) | | | | |
| STAreaSd | Stand. Dev. of ST segment | 0.06 | 0.52 | −0.10 | −0.05 |
| | area (mV s) | | | | |

PCA was employed to reduce the statistical redundancy between the high dimensional ECG features without significant loss of information. The application of PCA suggested that the first four principal components could explain approximately 78% of the variations in the data, and thus these four were considered for further studies. None of the other components captured more than 5% of the variation in the feature values. Table 1 shows the descriptions of 15 features and the contributions of each feature in four principal eigen directions. The larger the coefficients, the higher the contribution of that feature to the variation along that eigen direction. It is evident from Table 1 that all except the RR standard deviation (RR Std) and the difference ST-PR contributed to one of the leading components. The remaining 13 significant ECG features were used as predictors in the training and simulation phases.

In the training phase of this embodiment, the coefficient vectors of the empirical regression model were estimated offline. First, the pattern search (optimization) method described herein was used to tune the parameters pertaining to the elastance (Eₗᵥₛ, E_{lvd}, Eᵣᵥₛ, E_{rvd}), the offsets and gains of pressures (Gₚₐ, Oₚₐ, Gᵣₐ, Oᵣₐ, Gₚᵥ, Oₚᵥ), and the respiration coupling γ. The tuned parameters were regressed offline with selected ECG features to estimate the empirical regression model. Table 2 lists the significant ECG features (with p-value <10⁻³) and the corresponding coefficients of the empirical regression model. The subsequent simulation phase involved generating the hemodynamic signals in real time from the parameterized model. The model parameters in the simulation phase were estimated using the regression model with the selected ECG features as predictors.

TABLE 2
COEFFICIENTS OF REGRESSION MODEL TO ESTIMATE MODEL
PARAMETERS FROM ECG FEATURES

| | Feat. | | | | | | | | | | | | | |
|---|---|---|---|---|---|---|---|---|---|---|---|---|---|---|
| | | | | | RR | PR | ST | QT | ST | PR | QT | | ST | ST |
| Par | Intercept | R AmpAvg | P AmpAvg | T AmpAvg | Avg | Avg | Avg | Avg | Std | Std | Std | QT-PR | Area | AreaStd |
| Gₚₐ | 3.24 | | | | | | 0.3 | | −0.22 | 0.7 | | | | |
| Oₚₐ | 2.35 | 0.52 | | −0.1 | 0.68 | | | | | | | | | |
| Gᵣₐ | 0.4 | | | 0.1 | | | | −0.21 | | | | | 0.3 | −0.14 |
| Oᵣₐ | −0.3 | | | | | 0.25 | −0.18 | | | | | | | |
| Gₚᵥ | 4.35 | | | 0.91 | | | | 0.12 | 0.25 | | | | −3.23 | |
| Oₚᵥ | 10.51 | 1.14 | | | | −0.26 | | | | | | −3.16 | | |
| γ | 1.32 | −2.8 | 0.12 | | | | | | | 0.45 | | | 1.37 | −1.48 |
| Eₗᵥₛ | 0.94 | | 0.25 | 0.12 | | | −1.15 | 1.35 | | | | | 0.25 | 0.25 |
| Eᵣᵥₛ | 0.92 | | 0.2 | | | 3.19 | | −3.29 | | | −0.97 | | 1.72 | |
| E_{lvd} | 0.12 | | 0.19 | | | | | | 2.41 | −2.31 | | −0.35 | | 0.41 |
| E_{rvd} | 0.09 | 0.15 | | | | | | | 0.14 | | | −0.13 | | |

Next, and continuing with the present example, the salient hemodynamic signals extracted from the model outputs were compared with the actual measurements in the database. The following combinations of signals were compared:
i) Chamber pressures and volumes with ideal profiles.
ii) Right atrial pressure (Pᵣₐ) from the model with the measured central venous pressure (CVP).
iii) Pulmonary arterial pressures from the model (Pₚₐ) with those from actual measurements (PAP).
Pulmonary vein pressure from the model (Pₚᵥ) with the measured respiratory impedance (RI).
v) Systolic and diastolic (max and min) pressure values estimated from the model versus measurements.

The various patterns of the model-generated waveforms of pressures (FIG. 6) and volumes (FIG. 7) of the four heart chambers lay within similar ranges of values as those of ideal profiles. For the left ventricle, the pressure ranged from 0 to 125 mmHg, the volume varied between 70-130 ml, and the shapes (e.g., skewness and support) of the pressure and the volume waveform patterns are also comparable with those of the ideal profiles.

H1) Pulmonary arterial pressure comparisons

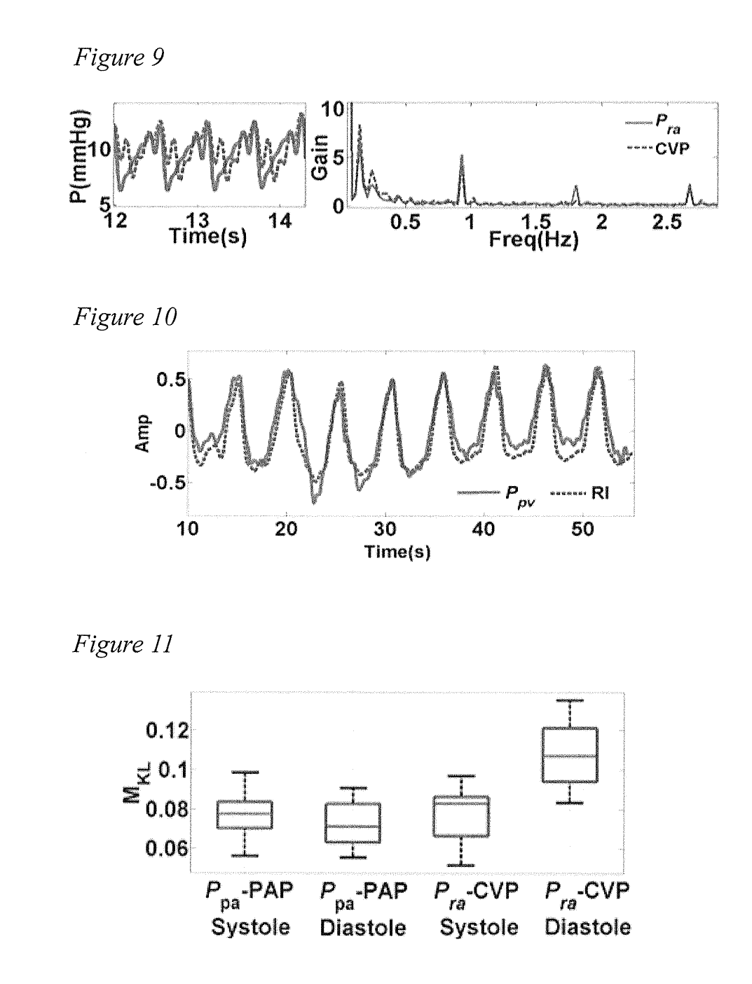

FIG. 9 shows the measured pulmonary arterial pressure (PAP) and the model output (Pₚₐ) waveforms in time (left) and frequency (right) domains. In the time domain, the pulmonary arterial pressure values range from 10 mHg through 30 mHg for both measured and model-generated waveforms and exhibit similar patterns, including the skewness and rise and drop rates. The frequency spectrum portrait shows the presence of respiratory and heart rate components at 0.15 Hz, and 0.93 Hz, respectively for both measured and model-derived data. Since PAP is measured at the phlebostatic axis, which is found at the intersection of the midaxillary line and a line drawn from the fourth intercostal space at the right side of the sternum on the thorax, the respiration is also included. These similarities between the pulmonary arterial pressure waveforms suggest that the model-generated Pₚₐ signals can be a suitable surrogate for PAP.

H2) Right Atrial Pressure and Central Venous Pressure Comparisons

According to an embodiment, the model-generated right atrial pressure (Pᵣₐ) waveform was compared with the measured central venous pressure (CVP) waveform. Since CVP captures the blood pressure in the thoracic vena cava near the atrium of the heart, it is considered a reasonable surrogate of Pᵣₐ. FIG. 9 shows the variation of CVP and Pᵣₐ in time (left) and frequency (right) domains. It may be noted that the Pᵣₐ waveform captures the amplitudes, and the time-domain patterns including the skewness, rise and drop rates of the CVP waveform. The frequency portraits were almost identical for both waveforms, with the frequency peaks of respiratory components at 0.15 and 0.24 Hz, and heart-rate components at 0.93 Hz. The similarities between the signal waveforms in both the time and frequency suggest that Pᵣₐ derived from the model output may serve as a viable surrogate for CVP.

H3) Pulmonary Vein Pressure and Respiratory Impedance Comparisons

A comparison of the waveforms of the low frequency 0.3 Hz) component of the pulmonary vein pressure (Pₚᵥ) and the respiratory impedance (RI) measurement in the time domain is shown in FIG. 10 for a particular embodiment. The time-domain patterns contained in the low frequency component of the Pₚᵥ waveform, including the respiratory components, are consistent with those of the RI, the change in the chest movement during the respiratory process. The Pₚᵥ captures the pressure of blood returning from the lung to the left atrium of the heart. As the elastance of the thorax can be assumed to be constant, the low frequency of the pulmonary venous pressure can be used for the measurement of the respiratory volume. Evident from the figure is that the low frequency components of the model-generated Pₚᵥ signal can be used to approximate the RI measurement.

Correlation coefficients ρ and R² statistic values were calculated to quantitatively assess the correlations between the model-derived and measured signals (Pₚₐ vs. PAP, Pᵣₐ vs. CVP, and Pₚᵥ vs. RI). A 10-fold cross validation was used to evaluate the accuracy of the model with an independent dataset. First, the dataset was randomly partitioned into 10 subsets, one of which was used for testing and other nine to build the parameter regression model. For model validation, the ECG features of the testing subset were used to estimate the model parameters. This process of partitioning followed by training and testing was repeated 20 times. Table 3 summarizes the average ρ and R² values of the various model-derived vs. measured waveforms. It is observed that the Pₚᵥ waveform matches well with the RI's with ρ=0.77 and R²=0.79. The correlations between other model-derived signals and the actual measurements are substantial (ρ≧0.68, and R²≧0.70).

TABLE 3
MODEL-DERIVED VS. MEASURED WAVEFORM
COMPARISONS

| Signal waveform | | |
|---|---|---|
| comparisons | ρ | R² |
| Pₚₐ vs. PAP | 0.71 | 0.73 |
| Pᵣₐ vs. CVP | 0.68 | 0.70 |
| Pₚᵥ vs. RI | 0.77 | 0.79 |

H4) Systolic and Diastolic Pressure Comparisons

A two-sample Anderson-Darling goodness-of-fit hypothesis test and KL divergence were used to evaluate the similarity of the systolic and diastolic values from the model outputs with those from the measured pressure profiles determined according to the present embodiment. During each heartbeat, the blood pressure varies between the maximum (systolic) and minimum (diastolic) values. Four sets of systolic (upper tail) and diastolic (lower tail) pressure samples were obtained from the pressure value distribution, each obtained by considering the 4th, 5th, 6th, or the 7th percentile (4-7% of) the observed signal realizations. The choice of extreme values (4-7%) was determined based on prior results that suggested that the augmentation (extreme) portions of an atrial pressure waveform are roughly 5% of the pulse pressure (i.e., waveform amplitude) for heart rates in the 60-110 beat/min range. Table 4 summarizes the rejection rate (at significance level α=0.05) of the Anderson-Darling test with the null hypothesis that the tails of the model-derived pressure waveforms and those of the measured pressure waveforms emerge from the same distributions. It is noted that for most of the comparisons the rejection rates are below 10%. These low rejection rates, in the 4-7% cut-off range, further indicate that the systolic and diastolic pressures of PAP and CVP can be statistically captured by the model-generated Pₚₐ and Pᵣₐ waveforms, respectively.

Next, KL information (M_{KL}) was used to quantify the difference between the distributions of the extrema of the pressure waveforms from the model outputs and those from the measurements. FIG. 11 shows the variation of the M_{KL} of the systolic and diastolic pressures of the model-derived signals and those of the actual measurements at a threshold of 5%. It is noted that the average KL information of the model-generated systolic and diastolic samples vs. those from recorded pressures are below 0.14. Such low values of KL information suggest that systolic and diastolic pressures from the model are comparable to those from actual measurements.

TABLE 4
AVERAGE REJECTION RATES FROM
ANDERSON-DARLING TEST

| | | |
|---|---|---|
| | Pulmonary | Central |
| | Arterial | Venous |
| | Pressure vs. | Pressure |
| | Pulmonary | vs. |
| Extreme | Arterial | Right Atrial |
| value | Pressure | Pressure |

| cut-off | Systole | Diastole | Systole | Diastole |
|---|---|---|---|---|
| 4% | 5% | 5% | 5% | 10% |
| 5% | 0 | 0% | 5% | 10% |
| 6% | 5% | 5% | 5% | 10% |
| 7% | 10% | 10% | 10% | 15% |

Further to the methodology described above, a Simulink/Matlab block diagram of the cardiovascular system is provided in the upper portion of FIG. 5. As can be seen, in this example, the diagram consists of three main components: the heart, systemic circulation loop, and pulmonary circulation loop. The heart is modeled as four chambers driven by ECG-based activation functions and four heart valves that control the blood flow direction. The systemic and pulmonary circulations are the combination of in the aortic sinus, artery, and vein segments. The descriptions of the Simulink block diagram of all cardiovascular system components are provided in the following paragraphs.

Heart Chambers

The same set of differential equations were used to model four heart chambers (i.e., left ventricle, right ventricle, left atrium, and right atrium). For example, the elastance of the left ventricle is taken to be a function of the characteristic elastances (Eₗᵥₛ and E_{lvd}), ventricular activation function, and respiration:

eₗᵥ(t)=E_{lvd}+(Eₗᵥₛ−E_{lvd})×(uᵥ(t)+γResp(t))

The pressure and volume of the ventricles are given as:

$$\frac{d {V}_{l v}}{d t} = {CQ}_{ao} {N}_{ao} \sqrt{\vert {P}_{l v} - {P}_{sas} \vert}$$

$${P}_{l v} = {P}_{l v , 0} + {e}_{l v} ( {V}_{l v} - {V}_{l v , 0} )$$

Heart Valves

According to an embodiment, the heart valves can be modeled by considering the effects of the blood pressure, tissues friction, and blood flow on the valve leaflets as shown in FIG. 14. The opening angle of the aortic valve is described as:

$$\frac{{d}^{2} {θ}_{ao}}{d {t}^{2}} = ( {P}_{lv} - {P}_{sas} ) {K}_{p , ao} cos {θ}_{ao} - {K}_{f , ao} \frac{d {θ}_{ao}}{d t} + {K}_{b , ao} {Q}_{ao} cos {θ}_{ao} - {K}_{v , ao} {Q}_{ao} ( μ ( {Q}_{ao} ) sin {θ}_{ao} + μ ( - {Q}_{ao} ) cos {θ}_{ao} )$$

$${Q}_{ao} = \{ \begin{matrix}{CQ}_{ao} \cdot {AR}_{ao} \cdot \sqrt{{P}_{lv} - {P}_{sas ,}} {P}_{lv} \geq {P}_{sas} , \\ {CQ}_{ao} \cdot {AR}_{ao} \cdot \sqrt{{P}_{sas} - {P}_{lv} ,} {P}_{lv} < {P}_{sas}\end{matrix}$$

Blood Circulation Loops

The systemic circulation loop is divided into five parts of aortic sinus, artery, arterioles, capillary, and vein. The aortic sinus and artery are quite elastic. Arterioles and capillaries are dominated by resistance effects. Vein functions to collect and store blood, thus resistance and compliance effects are considered in the vein model.

Great pressure and flow rate oscillations are experienced in the aortic sinus due to the local tissue elastance and flow variations. In an embodiment, the pressure is governed by

$$\frac{d {P}_{sas}}{d t} = \frac{{Q}_{ao} - {Q}_{sas}}{{C}_{sas}}$$

And the flow rate is

$$\frac{d {Q}_{sas}}{d t} = \frac{{P}_{sas} - {P}_{sat} - {R}_{sas} {Q}_{sas}}{{L}_{sas}}$$

The pressure and flow rate changes in the artery are similar to those in the aortic sinus. As arterioles and capillaries are both considered as pure resistance units, their effects are integrated with the artery as resistance units. Thus, the pressure equation is

$$\frac{d {P}_{sat}}{d t} = \frac{{Q}_{sas} - {Q}_{sat}}{{C}_{sat}}$$

And the flow rate equation is

$$\frac{d {Q}_{sat}}{d t} = \frac{{P}_{sas} - {P}_{svn} - ( {R}_{sas} + {R}_{sar} + {R}_{scp} ) {Q}_{sat}}{{L}_{sat}}$$

In this embodiment, the systemic vein is modeled as a compliance unit combined with a resistance unit. In the vein the pressure can be represented as:

$$\frac{d {P}_{svn}}{d t} = \frac{{Q}_{sat} - {Q}_{svn}}{{C}_{svn}} .$$

And the flow rate is given by

$${Q}_{svn} = \frac{{P}_{svn} - {P}_{ra}}{{R}_{svn}} .$$

The model of the pulmonary loop in some embodiments can be expressed in a manner that is similar to that of the systemic loop, with different values for system parameters. Great pressure and flow rate oscillations are experienced in the pulmonary valve due to the local tissue elastance and flow variations. The pressure is governed by:

$$\frac{d {P}_{pa}}{d t} = \frac{{Q}_{po} - {Q}_{pa}}{{C}_{pas}}$$

And the flow rate is

$$\frac{d {Q}_{pa}}{d t} = \frac{{P}_{pa} - {P}_{pat} - {R}_{pa} {Q}_{pa}}{{L}_{pas}}$$

Similarly, in some embodiments the pressure and flow rate changes in the artery are similar to those in the aortic sinus. As arterioles and capillaries are both considered as pure resistance units, their effects are integrated with the artery as resistance units. Thus, the pressure equation is:

$$\frac{d {P}_{pat}}{d t} = \frac{{Q}_{pa} - {Q}_{pat}}{{C}_{pat}}$$

And the flow rate equation is

$$\frac{d {Q}_{pat}}{d t} = \frac{{P}_{pa} - {P}_{pvn} - ( {R}_{sas} + {R}_{par} + {R}_{pcp} ) {Q}_{pat}}{{L}_{pat}}$$

The pulmonary vein can be modeled as a compliance unit combined with a resistance unit. In the vein the pressure might be written as

$$\frac{d {P}_{pvn}}{d t} = \frac{{Q}_{pat} - {Q}_{pvn}}{{C}_{pv}}$$

And the flow rate can be expressed as:

$${Q}_{pvn} = \frac{{P}_{pvn} - {P}_{la}}{{R}_{pv}}$$

See the exploded view of the box corresponding to the left atrium in FIG. 5. See additional examples of heart valve and blood circulation loops models developed by the differential equation and formulas of the pressure and flow rate oscillations set out in FIGS. 15-17.

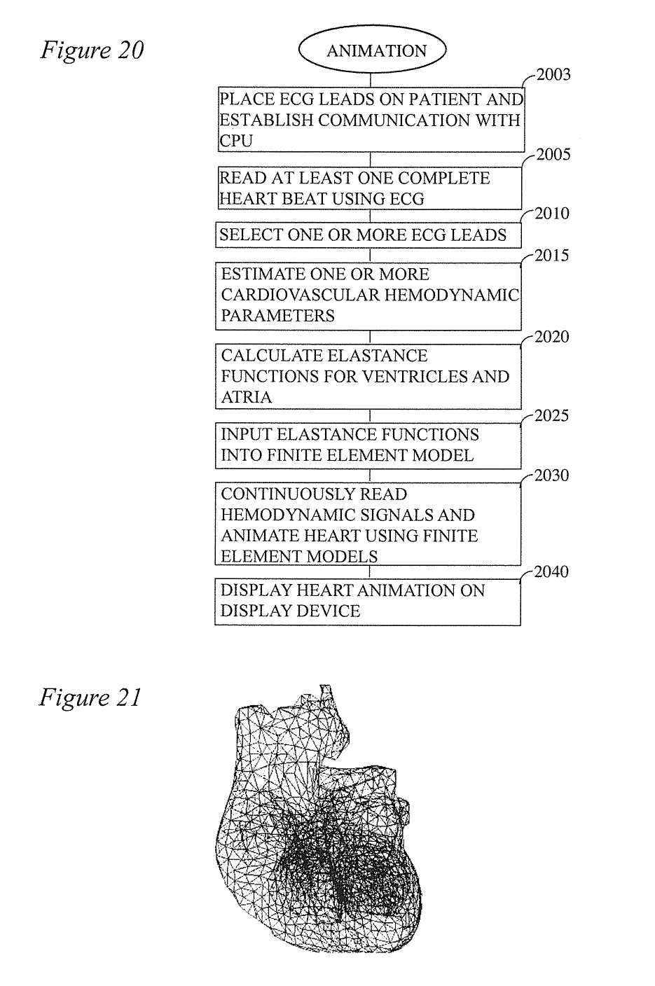

Turning next to FIG. 20, this figure contains an example operating logic suitable for use with a real time 3D heart animation embodiment of the instant disclosure, synchronized by the volume and pressure from the model.

According to the embodiment of FIG. 20, as a first step 2003 the ECG lead or leads will be affixed to (or otherwise placed into communication with) a patient. As has been discussed previously, in some embodiments only a single one of the ECG leads will be utilized in the steps that follow. Additionally, a communication link will need to be established with one or more CPUs which will be tasked with providing real-time animation. Note that the term “CPU” should be broadly interpreted to be a microprocessor, microcontroller, or other programmable device embedded in custom hardware. Additionally CPU could refer to a conventional desktop or other personal computer. Additionally, it should be understood that multiple CPUs might be involved in a particular instance, e.g., via networking. Thus CPU should be understood to be either singular or plural, as the context requires.

As a next step, the CPU will read and digitize at least one complete heart beat using the ECG hardware (step 2005). A purpose of this step is to obtain data that will be useful in the calculation of the cardiovascular hemodynamic parameters. In some embodiments, ten seconds of the ECG signal will be read and used in the calculation. However, the instant inventors have determined that normally the calculated values of the parameters will begin to stabilize after a few heartbeats. That being said, the particular number of seconds recorded (and the associated number of heartbeats captured) may need to be determined on a case-by-case basis, such a determination being well within the capability of a person of ordinary skill in the art.

Next, at least one ECG lead will be selected (step 2010). As has been explained previously, in some embodiments Lead II along has proven to be useful in the steps that follow. However, in some cases more than one lead might be used as has been described previously.

According to an embodiment, the data from the selected lead(s) will be used to calculate one or more of the cardiovascular hemodynamic parameters (step 2015) according to the equations set out above. That being said, it is possible that additional or different parameters might be calculated using the approach generally set out above.

Continuing with this example, next the elastance functions for the ventricles and atria will be calculated as discussed previously (step 2020). These functions will then be used as input into the finite element model of the heart (step 2025) as described previously.

Finally with respect to the current example, real-time cardiac signals including blood pressures, volumes, blood flow-rates within 4 chambers and heart valves will then continuously be read and the animation of the 3D heart synchronized by the derived volumes and pressures will be displayed in real-time as has been described above (step 2030). In some embodiment the 3D display will be presented to the user on a computer monitor that is in electronic communication with the computer(s) responsible for collecting the ECG data, calculating the hemodynamic parameters, evaluation the FEM model, and converting same into a displayable form for presentation to an attending user such as a physician or medical technician. The display might also be a stand-alone device that is in electronic communication with the computer(s) (e.g., connected via WiFi, Bluetooth, etc). In other instances, that animation might be projected (e.g., using an LED projector), viewed in simulated 3D (using red/blue glasses, polarized glasses, etc.), rotated automatically or under control of a user, etc. In short, any known or hereinafter developed method of visualizing a 3D heart model would be suitable for use with the instant invention.

This document teaches an approach to generating multiple synchronized hemodynamic signals from cardiovascular systems in real time using ECG features. ECG features are used to construct atrioventricular excitation inputs to a nonlinear deterministic lumped parameter model of cardiovascular system dynamics. Important events of the ECG signal are extracted by using wavelet analysis and a phase space method. Respiration components extracted from ECG play an important role in the representation of the model outputs. It is noteworthy that the model outputs were correlated substantially with the measured signals when the activation function included the respiration components. The salient time and frequency patterns of the model outputs, including right atrial pressure (Pᵣₐ), pulmonary artery pressure (Pₚₐ), and the low frequency components of pulmonary vein pressure (Pₚᵥ) were statistically consistent with those of actual recordings. The waveforms of Pₚₐ, Pᵣₐ and Pₚᵥ were substantially correlated with those of PAP, CVP and RI with R² of 0.73, 0.7, and 0.79, respectively. In addition, the results from a two-sample Anderson-Darling hypothesis test and KL information comparisons suggest strong statistical similarities between the distributions of model-derived and measured diastolic and systolic pressure values.

The similarities of the time and frequency domain characteristics as well as the statistically indistinguishable distribution of the tails of the model outputs and the measured waveforms indicate the suitability of using ECG features to form the excitation inputs for the cardiovascular model. Apart from the accuracy of the model outputs, the causal relationship captured in the model between the patterns of ECG and certain hemodynamic signals can provide valuable information for diagnosis because certain pathologies not easy to diagnose from the ECG patterns may be enhanced in the model outputs, such as pressure, volume, and blood flow rate signals. Further, the model provides an efficient tool for quantitatively assessing the underlying couplings between the mechanical and electrical components of heart dynamics and an ECG. It can also be used to simulate certain hemodynamic signals under specific cardiac disorders for each individual heart and to analyze the dependencies among ECG and other signal features and pathologies. Taken together, the results point to the viability of a virtual cardiovascular instrument platform where the derived hemodynamic signals can be used for clinical diagnosis and treatment. Such a platform can offer the advantages of cost efficiency and time savings.

The current model uses ECG alone to extract multiple hemodynamic signals. The ECG recordings may not effectively capture certain aspects of cardiovascular dynamics, especially those related to systemic and pulmonary circulations that determine the patterns of various hemodynamic signals. Furthermore, the model uses highly simplified expressions to model the effects of vascular components of cardiovascular regulation and hemodynamics and mostly ignores the effects of the nervous system and the kidney in rapid and long term control of arterial pressures. The model also does not contain explicit descriptions of the neural control scheme (e.g., autonomic nervous control of heat rate, myocardial contractility and vasomotor tone), hormones, volume receptors, and metabolism that might play important roles in characterizing the cardiovascular system dynamics. Since the dynamics of the cardiovascular system are characterized as a lumped parameter model where the model components (e.g., heart chambers, heart valves, arteries, arterioles, capillaries, and vein segments) are treated as homogeneous elements, the model is not applicable in modeling the heart with localized disorders. To further improve the model's accuracy, it is necessary to improve the structures of the dynamics model by involving other important effects of the circulatory hemodynamics, pulmonary mechanics, and ventilator control, as well as using more generalized regression models for parameter estimation. Also, the suitability of the model for characterizing the complexity of the human cardiovascular system (homeostatic processes, adaptive control of heart rate, and varying degrees of contractility) and to capture the dynamics of larger sets of individuals (e.g., age groups, genders, disorders, and medication conditions) remains to be investigated. A more comprehensive visualization and quantification of the heart, hemodynamic couplings, and their relationship with ECG and other signal features can lead to more responsive and cost-effective healthcare delivery.

For purposes of the instant disclosure, the term “real time” should be understood to refer to processing and/or display of information at the same rate that it is received and contemporaneous with the receipt of such data.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)−(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

It should also be noted that when the word “continuously” is used here (e.g., in connection with reading signals that originate from an ECG lead) that term should be understood to refer to repeated reading of a signal for some period of time. In some embodiments, “continuously” will refer to sampling at a rate of about 250 Hz, although higher or lower rates could also be used (e.g., a 2 kHz sample rate might be useful in some cases).

While this invention is susceptible of embodiment in many different forms, there is shown in the drawings, and is herein described in detail, some specific embodiments. It should be understood, however, that the present disclosure is to be considered an exemplification of the principles of the invention and is not intended to limit it to the specific embodiments or algorithms so described. Those of ordinary skill in the art will be able to make various changes and further modifications, apart from those shown or suggested herein, without departing from the spirit of the inventive concept, the scope of which is to be determined by the following claims.

APPENDIX

| Par | Initial Val | Unit | Par | Initial | Unit |
|---|---|---|---|---|---|
| Left heart | | | Right heart | | |
| α | 0.2 | | α | 0.2 | |
| CQₐₒ | 350 | ml/ | CQₚ | 350 | ml/(s |
| CQₘᵢ | 400 | ml/smmHg^{0.} | CQₜᵢ | 400 | ml/(s |
| Eₗᵥₛ | 2.5 | mmHg/ml | Eᵣᵥₛ | 1.15 | mmHg/ml |
| E_{lvd} | 0.1 | mmHg/ml | E_{rvd} | 0.1 | mmHg/ml |
| P_{lv,0} | 1 | mmHg | P_{rv,0} | 1 | mmHg |
| V_{lv,0} | 5 | ml | V_{rv,0} | 10 | ml |
| Eₗₐₛ | 0.25 | mmHg/ml | Eᵣₐₛ | 0.25 | mmHg/ml |
| E_{lad} | 0.15 | mmHg/ml | E_{rad} | 0.15 | mmHg/ml |
| P_{la,0} | 1 | mmHg | P_{ra,0} | 1 | mmHg |
| V_{la,0} | 4 | ml | V_{ra,0} | 4 | ml |
| Systemic Circulation | | | Pulmonary Circulation | | |
| Cₛₐₛ | 0.08 | ml/mmHg | Cₚₐₛ | 0.18 | ml/mmHg |
| Rₛₐₛ | 0.003 | mmHg s/ml | Rₚₐₛ | 0.002 | mmHg s/ml |
| Lₛₐₛ | 62 × 10⁻⁶ | mmHg s²/ml | Lₚₐₛ | 52 × 10⁻⁶ | mmHg s²/ml |
| Cₛₐₜ | 1.6 | ml/mmHg | Cₚₐₜ | 3.8 | ml/mmHg |
| Rₛₐₜ | 0.05 | mmHg s/ml | Rₚₐₜ | 0.01 | mmHg s/ml |
| Lₛₐₜ | 0.0017 | mmHg s²/ml | Lₚₐₜ | 0.0017 | mmHg s²/ml |
| Rₛₐᵣ | 0.5 | mmHg s/ml | Rₚₐᵣ | 0.05 | mmHg s/ml |
| R_{scp} | 0.52 | mmHg s/ml | R_{pcp} | 0.25 | mmHg s/ml |
| Rₛᵥₙ | 0.075 | mmHg s/ml | Rpv | 0.006 | mmHg s/ml |
| Cₛᵥₙ | 20.5 | ml/mmHg | Cₚᵥ | 20.5 | ml/mmHg |
| C_{svc} | 1.5 | ml/mmHg | C_{pvc} | 1.5 | ml/mmHg |
| Vₗᵥ₀ | 800 | ml | Vᵣᵥ₀ | 500 | ml |
| | | | Pulmonary and | | |
| Aortic and Mitral Valve | | | tricuspid Valves | | |
| K_{p,ao} | 5500 | rad/(s m) | K_{p,po} | 5500 | rad/(s m) |
| K_{f,ao} | 50 | 1/s | K_{f,po} | 50 | 1/s |
| K_{b,ao} | 2 | rad/(s m) | K_{b,po} | 2 | rad/(s m) |
| K_{v,ao} | 7 | rad/(s m) | K_{v,po} | 3.5 | rad/(s m) |
| K_{p,ml} | 5500 | rad/(s m) | K_{p,ti} | 5500 | rad/(s m) |
| K_{f,ml} | 50 | 1/s | K_{f,ti} | 50 | 1/s |
| K_{b,ml} | 2 | rad/(s m) | K_{b,ti} | 2 | rad/(s m) |
| K_{v,ml} | 3.5 | rad/(s m) | K_{v,ti} | 3.5 | rad/(s m) |

| | |
|---|---|
| Nomenclature | |
| C | compliance |
| e | elastance |
| K, k | coefficient |
| M | mass |
| P | pressure |
| Q | flow rate |
| V | volume |
| θ | phase angle |
| Subscripts used | |
| 0 | initial value, |
| 1, 2, . . . | subscript state |
| s | systole |
| d | diastole |
| lv | left ventricle |
| rv | right ventricle |
| la | left atrium |
| ra | right atrium |
| ao | aortic valve |
| mi | mitral valve |
| po | pulmonary aortic |
| ti | tricuspid valve |
| pas | pulmonary aortic |
| par | pulmonary arterioles |
| pat | pulmonary artery |
| pcv | pulmonary capillary |
| pvn | pulmonary vein |
| sas | systemic aortic sinus |
| sar | systemic arterioles |
| sat | systemic arterial |
| scv | systemic capillary |
| svn | systemic vein |
| sat | systemic arterial |

What is claimed is:

1. A method of displaying a real time 3D modeling of a patient's heart, comprising the steps of:
a. positioning at least one ECG lead to read ECG signals from the patient's heart;
b. selecting at least one of said at least one ECG lead;
c. using said selected at last one of said ECG lead to read patient ECG signals for a period of time at least as long as one complete heartbeat of the patient;
d. using said read patient ECG signals to estimate one or more cardiovascular hemodynamic parameters;
e. calculating elastance functions for a left ventricle, a right ventricle, a left atrium and a right atrium of the patient's heart;
f. continuously reading said selected at least one ECG lead;
g. while said selected at least one ECG lead is being read, using at least said elastance functions, said at least one cardiovascular hemodynamic parameters and said read selected at least one ECG lead to animate and display in real-time a heart model of the patient's heart.

2. A method according to claim 1, wherein exactly one ECG lead is selected.

3. A method according to claim 2, wherein said selected ECG lead is lead II.

4. A method according to claim 1, wherein said one or more cardiovascular hemodynamic parameters are selected from the group consisting of Eₗᵥₛ, Eᵣᵥₛ, E_{lvd}, E_{rvd}, γ, Gₚₐ, Oₚₐ, Gᵣₐ, Oᵣₐ, Gₚᵥ, and Oₚᵥ, where:
Eₗᵥₛ, is a first elastance characteristic of the left ventricle,
Eᵣᵥₛ is a second elastance characteristic of the left ventricle,
E_{lvd} is a first elastance characteristic of the right atrium,
E_{rvd} is a second elastance characteristic of the right atrium,
γ is a parameter representative of respiratory coupling,
Gₚₐ is a parameter that quantifies a gain associated with a pulmonary arterial pressure,
Oₚₐ is a parameter that quantifies an offset associated with a pulmonary arterial pressure,
Gᵣₐ is a parameter that quantifies a gain associated with a right arterial pressure,
Oᵣₐ is a parameter that quantifies an offset associated with a right venous pressure,
Gₚᵥ is a parameter that quantifies a pressure associated with a pulmonary venous pressure, and,
Oₚ, is a parameter that quantifies an offset associated with a pulmonary venous pressure.

5. A method according to claim 1 wherein said calculated elastance functions for the left ventricle, the right ventricle, the left atrium and the right atrium of the patient's heart are given by the equations:
eₗₐ(t)=E_{lad}+(Eₗₐₛ−E_{lad})*(uₐ)+γResp(t))
eₗᵥ(t)=E_{lvd}+(Eₗᵥₛ−E_{lvd})*(uᵥ(t)+γResp(t)),
eᵣₐ(t)=E_{rad}+(Eᵣₐₛ−E_{rad})*(uₐ)(t)+γResp(t))
eᵣᵥ(t)=E_{rvd}α(Eᵣᵥₛ−E_{rvd})*(u_{c}(t)+γResp(t)).

6. A method according to claim 1, wherein said heart model of the patient is a finite element model.

7. A method of estimating one or more cardiovascular hemodynamic parameters representative of an aspect of a patient's heart, comprising the steps of:
a. positioning at least one ECG lead to read ECG signals from the patient's heart;
b. selecting at least one of said at least one ECG lead;
c. using said selected at last one of said ECG lead to read patient ECG signals for a period of time at least as long as one complete heartbeat of the patient;
d. using said read patient ECG signals to estimate one or more cardiovascular hemodynamic parameters;
e. using any of said estimated one or more cardiovascular hemodynamic parameters to determine a condition of the patient's heart.

8. A method according to claim 7, wherein step (e) comprises the steps of:
e. comparing each of said one or more of said estimated cardiovascular hemodynamic parameters with a population norm for said each of said estimated cardiovascular hemodynamic parameter, thereby determining a condition of the patient's heart.

9. A method according to claim 7, wherein exactly one ECG lead is selected.

10. A method according to claim 9, wherein said selected ECG lead is lead II.

11. A method according to claim 1, wherein said one or more cardiovascular hemodynamic parameters are selected from the group consisting of Eₗᵥₛ, Eᵣᵥₛ, E_{lvd}, E_{rvd}, γ, Gₚₐ, Oₚₐ, Gᵣₐ, Oᵣₐ, Gₚᵥ, and Oₚᵥ, where:
Eₗᵥₛ, is a first elastance characteristic of the left ventricle,
Eᵣᵥₛ is a second elastance characteristic of the left ventricle,
E_{lvd} is a first elastance characteristic of the right atrium,
E_{rvd} is a second elastance characteristic of the right atrium,
γ is a parameter representative of respiratory coupling,
Gₚₐ is a parameter that quantifies a gain associated with a pulmonary arterial pressure,
Oₚₐ is a parameter that quantifies an offset associated with a pulmonary arterial pressure,
Gᵣₐ is a parameter that quantifies a gain associated with a right arterial pressure,
Oᵣₐ is a parameter that quantifies an offset associated with a right venous pressure,
Gₚᵥ is a parameter that quantifies a pressure associated with a pulmonary venous pressure, and,
Oₚᵥ is a parameter that quantifies an offset associated with a pulmonary venous pressure.
